(19) United States
(12) Patent Application Publication
(10) Pub. No.: US 2024/0042175 A1
(43) Pub. Date: Feb. 8, 2024

(54) MEDICAL DEVICES INCORPORATING SENSORS, SYSTEMS, AND RELATED METHODS

(71) Applicant: XENTER, INC., Draper, UT (US)

(72) Inventor: Richard J. LINDER, Sandy, UT (US)

(21) Appl. No.: 18/229,044

(22) Filed: Aug. 1, 2023

Related U.S. Application Data
(60) Provisional application No. 63/394,584, filed on Aug. 2, 2022.
(60) Provisional application No. 63/394,591, filed on Aug. 2, 2022.

(51) Int. Cl. A61M 25/02

(52) U.S. Cl. CPC A61M 25/02; A61M 2025/0001

(57) ABSTRACT

Medical devices, such as catheters, may include sensors for detecting or measuring desired parameters. In a related method, a flexible, elongate member is inserted into a vein of a patient. The distal end of the flexible, elongate member is positioned near a cavoatrial junction of the patient (e.g., within the superior vena cava). A fluid is passed through a lumen of the flexible, elongate member and through an exit port of the flexible, elongate member. A temperature of the fluid may be determined using a first sensor associated with the flexible, elongate member, and then amount of time that it takes for a second sensor to record a temperature that correlates with the determined temperature of the fluid is measured. A flow characteristic may then be determined based on a known distance between the first sensor and the second sensor and the measured time period.

### CROSS-REFERENCE TO RELATED APPLICATIONS

The present application claims the benefit of U.S. Provisional Patent Application No. 63/394,584 entitled MEDICAL DEVICES INCORPORATING SENSORS, SYSTEMS, AND RELATED METHODS, filed on Aug. 2, 2022, and U.S. Provisional Patent Application No. 63/394,591 entitled MEDICAL DEVICES, SYSTEMS, AND METHODS INCLUDING POWER AND DATA TRANSFER, filed on Aug. 2, 2022, the disclosures of which are incorporated by reference herein in their entireties.

### BACKGROUND

The present invention relates generally to medical devices incorporating sensors, as well as systems incorporating such devices and related methods. In one, non-limiting example, such medical devices may include intraluminal devices, such as catheters, that include various sensors for measuring of one or more physiological parameters and/or for imaging a patient's anatomy.

In one example, it may be desirable (in a variety of circumstances) to determine the cardiac output of a patient. Conventionally, this will include subjecting a patient to what is known as a Swan-Ganz catheterization (also referred to as a right heart catheterization or pulmonary artery catheterization). This procedure is conducted by a physician, conventionally in an intensive care unit or in a cardiac catheterization lab. Often, such a procedure is administered using a sedative. During the procedure, the doctor will insert a catheter in a patient's arm or leg and guide the catheter up into the patient's heart. X-ray images are often used to guide the catheter to the proper location. An electrocardiogram is administered to monitor the patient's heart during the procedure.

The Swan-Ganz catheterization procedure, while effective, is rather invasive and is a fairly extensive procedure requiring significant resources in time, personnel, dedicated space, and dedicated medical devices.

As with many areas of medicine, there is a continued desire to provide more efficient approaches to diagnosing and treating patients. This includes a need for improved devices and systems that enable procedures to be conducted, for example, more efficiently and/or less invasively.

### SUMMARY

The present disclosure provides medical devices, medical device systems, and related methods. In some embodiments, such devices and systems provide the ability to detect or measure a parameter at or near the cavoatrial junction of a patient. In some embodiments, the devices and/or systems may enable a practitioner to determine (and/or monitor) the cardiac output of a patient without having to place the patient in a cardia catheterization lab and without requiring a physician to perform the procedure (e.g., the procedure could be performed by a trained nurse or other practitioner).

In one embodiment, a medical device comprises a flexible, elongated member configured for insertion within a body, the flexible, elongated member having a proximal end, a distal end, and at least one lumen extending along a length of the flexible, elongated member. A first temperature sensor is disposed adjacent an exit port, the exit port being in fluid communication with the at least one lumen. A second temperature sensor disposed a known distance from the first temperature sensor.

In one embodiment, the medical device further comprises at least one additional sensor.

In one embodiment, the at least one additional sensor comprises a pressure sensor.

In one embodiment, the flexible, elongated member is configured as at least one of a peripherally inserted central catheter (PICC) or a central venous catheter.

In one embodiment, the first temperature sensor and the second temperature sensor are in wireless electrical communication with an external device.

In one embodiment, the electrical communication of the first temperature sensor and the second temperature sensor includes providing power to the first temperature sensor and the second temperature sensor.

In another embodiment, a method of determining cardiac output of a patient is provided. The method includes: inserting a flexible, elongated member into a vein of a patient; locating a distal end of the flexible, elongate member near a cavoatrial junction of the patient; passing a fluid through a lumen of the flexible, elongated member and through an exit port of the flexible, elongated member; determining a temperature of the fluid using a first sensor associated with the flexible, elongated member; measuring a time period that it takes for a second sensor associated with the flexible, elongated member to record a temperature that correlates with the determined temperature of the fluid; and determining a flow characteristic based on a known distance between the first sensor and the second sensor and the measured time period.

In one embodiment, the method further comprises determining a fluid pressure using a third sensor associated with the flexible, elongated member.

In one embodiment, inserting a flexible, elongated member into a vein of a patient includes inserting at least one of a peripherally inserted central catheter (PICC) and a central venous catheter into the vein of the patient.

In one embodiment, the method further comprises leaving the flexible, elongated member in the vein of the patient and repeating the acts of passing a fluid, determining a temperature, measuring a time period, and determining a flow characteristic.

In one embodiment, leaving the flexible, elongated member in the vein of the patient includes leaving the flexible, elongated member in the vein of the patient for a period of at least a week.

In accordance with a further embodiment, a system is provided that comprises a medical device having: a flexible, elongated member configured for insertion within a body such that a distal end may be positioned at or near a cavoatrial junction of a patient, the flexible, elongated member having a proximal end, and at least one lumen extending along a length of the flexible, elongated member; and at least one sensor disposed at the distal end of the flexible, elongated member. The system further includes an external device in wireless communication with the medical device and configured to receive data from the at least one sensor.

In one embodiment, the at least one sensor includes at least one of a pressure sensor, a temperature sensor, a flow sensor, an oxygen sensor, and a glucose sensor.

In one embodiment, the at least one sensor includes a first temperature sensor and a second temperature sensor disposed a known distance from the first temperature sensor.

In one embodiment, wherein the flexible, elongated member is configured as a peripherally inserted central catheter (PICC).

In one embodiment, the flexible, elongated member is configured as a central venous catheter (CVC).

In one embodiment, the external device provides power to the at least one sensor.

In one embodiment, the external device provides wireless power to the at least one sensor.

In one embodiment, the system further comprises a volume of fluid in communication with lumen of the elongated, flexible member.

In one embodiment, the at least one sensor includes at least one temperature sensor and at least one pressure sensor.

Features, components, and aspects of one embodiment may be combined with features, components or aspects of another embodiment without limitation.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1 illustrates a catheter system according to an embodiment of the present disclosure;

FIG. 2 is an enlarged detail of a portion of the catheter shown in FIG. 1; and

FIG. 3 is a flow diagram illustrating a method according to an embodiment of the present disclosure.

### DETAILED DESCRIPTION

Various embodiments described herein are directed toward the incorporation of electronic devices (e.g., sensors and transducers) into medical devices and systems as well as related methods which may incorporate such medical devices and systems.

In some embodiments, devices associated with cardiovascular procedures are provided having sensors integrated therewith. For example, catheters may include sensors, transducers or other electronic or optical components integrated into the structure for detecting or measuring physiological data (e.g., pressure, flow rate, temperature, etc.), providing imaging data (e.g., ultrasound images), and provide that data on-demand or continuously in real time as may be required.

In some embodiments, other sensors or electronic elements are associated with the device. For example, sensors configured to detect the presence of biological components may be incorporated into or otherwise associated with the device. In some embodiments, an antenna structure may be integrated with the device for providing wireless transmission of data.

Some non-limiting examples of medical devices that may incorporate such sensors include those described in: U.S. patent application Ser. No. 17/205,964, entitled “Guidewire for Imaging and Measurement of Pressure and Other Physiological Parameters” and filed on Mar. 18, 2021; U.S. patent application Ser. No. 17/205,854, entitled “Catheter for Imaging and Measurement of Pressure and Other Physiological Parameters” and filed on Mar. 19, 2021; U.S. patent application Ser. No. 17/205,754, entitled “Operatively Coupled Data and Power Transfer Device for Medical Guidewires and Catheters with Sensor” and filed on Mar. 18, 2021; U.S. patent application Ser. No. 17/205,614, entitled “Signal Conducting Device for Concurrent Power and Data Transfer to and From Un-wired Sensors Attached to a Medical Device” and filed on Mar. 18, 2021; U.S. patent application Ser. No. 17/979,629, entitled “Data and Power Transfer Devices for Use with Medical Devices and Related Methods” and filed Nov. 2, 2022, the disclosures of which are each incorporated by reference herein in their entireties. Any of the components described in the foregoing references my be combined with the components and features described in the present application.

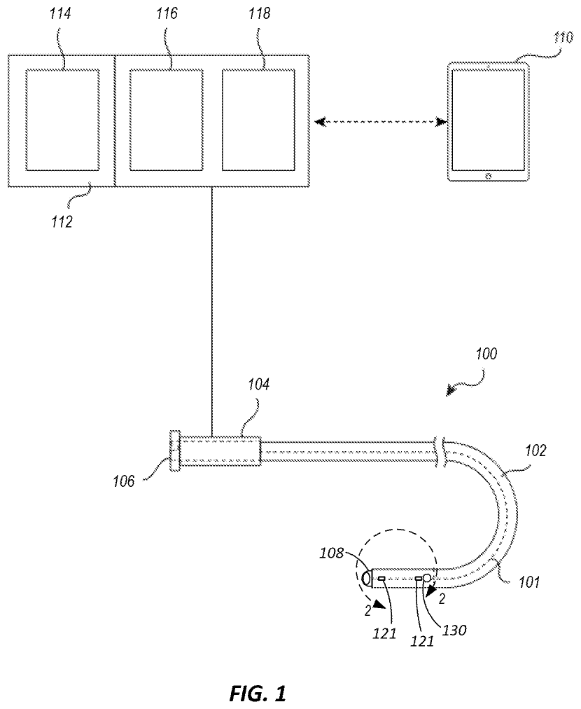

FIG. 1 is an overview of a catheter system 100 that may incorporate one or more of the features described herein. The catheter system 100 includes a catheter 102 that extends from a proximal end 106 to a distal end 108. The catheter 102 may include one lumen or multiple lumens. Typically, the catheter 102 will be formed at least primarily from one or more medical-grade polymer materials, though some embodiments may include other materials such as metals. For example, the term catheter 102, as used herein, can refer to other medical devices that comprise an elongate tube structure, having one or more lumens, configured for insertion into the body, including a hypotube or micromachined tube, unless specified otherwise. In some embodiments the catheter system 100 may include one or more nested catheters (e.g., arranged in a “telescoping” configuration). Separate layers may include, for example, braided layers, liners, polymer coatings, other catheter layers as known in the art, or combinations thereof.

The catheter system 100 may additionally include a proximal device 104 which, in some embodiments, includes a power and/or data coupling device. The proximal device 104 may also include or be associated with a transmitter to enable wireless communication between the catheter system 100 and an external device 110 (or multiple such external devices). The wireless system of the catheter system 100 may include, for example, a personal area network (PAN) (e.g., ultra-high frequency radio wave communication such as Bluetooth®, ZigBee®, BLE, NFC), a local area network (LAN) (e.g., WiFi), or a wide area network (WAN) (e.g., cellular network such as 3G, LTE, 5G). Wireless data transmission may additionally or alternatively include the use of light signals (infrared, visible radio, with or without the use of fiber optic lines), such as radiofrequency (RF) sensors, infrared signaling, or other means of wireless data transmission. In other embodiments, the catheter system 100 and external device 110 may be connected via a wired connection.

The external device 110 may be a hand-held device, such as a mobile phone, tablet, or lap-top computer. Although exemplary embodiments are described herein as using hand-held or mobile devices as the external devices 110, it will be understood that this is not necessary, and other embodiments may include other “non-mobile” devices such as a desktop computer, monitor, projector, or the like. In some embodiments, the external device 110 includes a mobile/hand-held device and additionally includes a desktop device or other non-mobile device. For example, a mobile device may be configured to receive transmitted data from the transmitter and function as a bridge by further sending the data to the non-mobile computer system. This may be useful in a situation where the physician would like the option of viewing data on a mobile device but may need to have the data additionally or alternatively passed or mirrored on a larger monitor such as when both hands are preoccupied (e.g., while handling the catheter system 100).

Processing of sensor data signals may be fully or primarily carried out at the external device 110, or alternatively may be at least partially carried out at one or more other external devices communicatively connected to the external device 110, such as at a remote server or distributed network. Additionally, or alternatively, sensor data signals may be processed at the coupling device 104, on the catheter 102, or at some combination of devices within the catheter system 100. Sensor data signals may include, for example, image data, location data, and/or various types of sensor data (as related to fluid flow, fluid pressure, presence/levels of various gases or biological components, temperature, other physical parameters, and the like).

The proximal device 104 includes a control unit 112 (shown enlarged and in schematic form) that includes a power source 114, data signal processor 116, and optionally a transmitter/receiver 118. The transmitter/receiver 118 enables wireless communication to the external device 110 (or multiple such devices) as described above with respect to FIG. 1.

The data signal processor 116 is configured to receive sensor data signals, sent through the catheter 102, from one or more sensors 121 associated with the catheter 102. The power source 114 is configured to transmit power through the catheter 102 to power the one or more sensors 121 and/or other components of the catheter 102. The power source 114 may include an on-board power source, such as a battery or battery pack, and/or may include a wired connection to an outside power source. The one or more sensors 121 may be located at any suitable position on the catheter 102 but will often be disposed at the distal section of the catheter 102 expected to reach the targeted anatomy.

Sensors 121 may be coupled to the catheter 102 (e.g., to the distal section of the catheter 102) by employing, for example, bonding, molding, co-extrusion, welding, and/or gluing techniques. Additionally, or alternatively, sensors 121 may be coupled to a substrate which itself is attached to the catheter 102, as explained in more detail below. As used herein, the “distal section” or “distal portion” refers to the distal-most 30 cm of the device, the distal-most 20 cm of the device, the distal-most 15 cm of the device, the distal-most 10 cm of the device, or to a range using any two of the foregoing values as endpoints. In some embodiments, the “intermediate section” may be considered as roughly the middle third of the device, and the “proximal section” or “proximal portion” may be considered as roughly the proximal third of the device.

In some embodiments, power and/or data lines 101 extend along the length of the catheter 102 to the one or more sensors 121. In some embodiments, one or more power and/or data lines 101 may be incorporated into or may form at least a portion of a braid structure within the catheter 102. As used herein, a “power line” and/or “data line” refer to any electrically conductive pathway (e.g., traces) within the medical device. Although multiple power and/or data lines 101 may be utilized, other embodiments may be configured to send both power and data on a single wire and/or manage sensor data signals from multiple sensors on a single wire. This reduces the number of wires that must be routed through (or along) the structure of the catheter 102 and more effectively utilizes the limited space of the device, as well as reducing the complexity of the device and the associated risk of device failure.

In some embodiments, multiple power and/or data signals (e.g., data signals from multiple sensors 121) can be sent through a common line 101 simultaneously. Power and/or data signals can also be sent in a “continuous” fashion. That is, the power and/or data signals can have a sufficiently high sampling rate such that the information is provided to the user within time frames that are practically “real-time”. For most applications, this will include sampling rates of approximately 5 seconds or less, 3 seconds or less, 1 second or less, or sub-second sampling rates.

In some embodiments, the proximal coupling device 104 may include one or more ports to facilitate the introduction of fluids (e.g., medications, nutrients) into the catheter 102 (e.g., a Tuohy-Borst adapter with a Y-connector). The catheter 102 may be sized and configured to be temporarily inserted in the body, permanently implanted in the body, or configured to deliver an implant in the body. In one embodiment, the catheter 102 is a peripherally inserted central catheter (PICC) line, typically placed in the arm or leg of the body to access the vascular system of the body. The catheter 102 may also be a central venous catheter (CVC), an IV catheter, coronary catheter, stent delivery catheter, balloon catheter, atherectomy catheter, or IVUS catheter or other imaging catheter. The catheter 102 may be a single or multi-lumen catheter.

The catheter system 100 may be effectively utilized in applications where localization of the distal section of the system would be beneficial. For example, localization features described in some of the previously incorporated references may be utilized to aid in stent delivery or proper placement of a PICC or CVC type catheter at a targeted site such as the cavoatrial junction.

The one or more sensors 121 of the catheter system 200 may include a pressure sensor, flow sensor, imaging sensor, temperature sensor, and/or a component detection sensor, for example. A pressure sensor (or multiple pressure sensors) may be sized and configured to sense changes in pressure in the environment. A flow sensor (or multiple flow sensors) may be sized and configured to sense the fluid flow, such as velocity or other flow characteristics. A detection sensor (or multiple detection sensors) may detect a proximity or distance to one or more detection nodes positioned external relative to the body or relative to one or more components positioned within the body. An imaging sensor may gather various forms of imaging data. A temperature sensor (or multiple temperature sensors) may be used to determine a temperature, or a change in temperature, of one or more components within a body.

The one or more sensors 121 may be additionally or alternatively be configured to sense the presence of biological components or measure physiological parameters in the targeted anatomical location (e.g., in the blood). Example biological components that may be detected/measured include sugar levels, pH levels, CO2 levels (CO2 partial pressure, bicarbonate levels), oxygen levels (oxygen partial pressure, oxygen saturation), temperature, and other such substrates and physiological parameters. The one or more sensors may be configured to sense the presence, absence, or levels of biological components such as, for example, immune system-related molecules (e.g., macrophages, lymphocytes, T cells, natural killer cells, monocytes, other white blood cells, etc.), inflammatory markers (e.g., C-reactive protein, procalcitonin, amyloid A, cytokines, alpha-1-acid glycoprotein, ceruloplasmin, hepcidin, haptoglobin, etc.), platelets, hemoglobin, ammonia, creatinine, bilirubin, homocysteine, albumin, lactate, pyruvate, ketone bodies, ion and/or nutrient levels (e.g., glucose, urea, chloride, sodium, potassium, calcium, iron/ferritin, copper, zinc, magnesium, vitamins, etc.), hormones (e.g., estradiol, follicle-stimulating hormone, aldosterone, progesterone, luteinizing hormone, testosterone, thyroxine, thyrotropin, parathyroid hormone, insulin, glucagon, cortisol, prolactin, etc.), enzymes (e.g., amylase, lactate dehydrogenase, lipase, creatine kinase), lipids (e.g., triglycerides, HDL cholesterol, LDL cholesterol), tumor markers (e.g., alpha fetoprotein, beta human chorionic gonadotrophin, carcinoembryonic antigen, prostate specific antigen, calcitonin), and/or toxins (e.g., lead, ethanol).

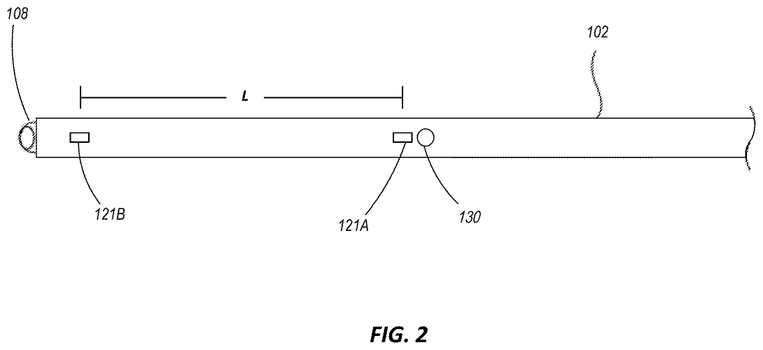

Referring now to FIG. 2, an enlarged view of a portion of a distal end of the catheter 102 is shown in accordance with an embodiment of the present disclosure. The catheter 102 may include an exit port 130 extending through a sidewall of the catheter at a location that is proximal of the distal end 108 of the catheter 102. A first sensor 121A may be positioned adjacent to (e.g., distally adjacent to) the exit port 130. A second sensor 121B may be positioned distal of the first sensor a known distance “L”. In one embodiment, the first and second sensors 121A and 121B may be configured as thermistors or temperature sensors. Additional sensors may also be located in the distal section of the catheter 102. For example, additional temperature sensors may be used, where the position of each sensor is known relative to each other. In other embodiments, other types of sensors may be used, including pressure sensors, flow sensors, oxygen sensors, or other biological component detecting sensors.

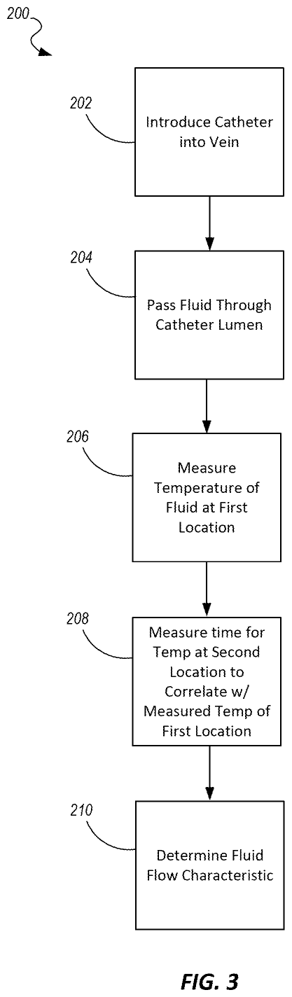

Referring to FIG. 3 with continued reference to FIGS. 1 and 2, a method 200 is shown regarding the use of the catheter 102. The method may include introducing the catheter 102 into a vein, as shown at 202, and placing the distal end 108 at a specified location within a patient's circulatory system (e.g., in the superior vena cava, at or near the cavoatrial junction). The catheter 102 may include, for example, a PICC or a CVC catheter (e.g., a catheter which is sized and configured to have its distal end positioned at or near the cavoatrial junction) and may be introduced, for example, through one of the internal jugular, femoral, subclavian, basilic or brachial veins. The catheter may be configured for long term placement (e.g., weeks or months) within the patient.

With the catheter 102 positioned as desired, a practitioner (e.g., a doctor, a nurse, a physician's assistant) may introduce a fluid through a lumen of the catheter 102, as indicated at 204, such that it flows through the exit port 130. This may be done as a matter of conventional practice, such as by flushing the catheter 102 (or a particular lumen of the catheter). As indicated at 206, the first sensor 121A may measure the temperature of the fluid as it passes from the exit port 130—the temperature of the fluid being different from the temperature of the patient's blood. As indicated at 208, the time for the passage of the fluid to flow from the first sensor 121A to the second sensor 121B (which sensor 121B will detect the fluid by recognizing the temperature becoming aligned with that which is measured during act 206) is monitored. Knowing the distance L between the first and second sensors 121A and 121B, and knowing the time that it took for fluid exiting the port 130 to pass from first sensor 121A and 121B, fluid flow characteristics may be calculated, as indicated at 210 to help determine cardiac output of the patient. The monitoring, measuring, and calculating acts may be accomplished using appropriate processors or circuitry such as, for example, may be disposed in the catheter 102, in the proximal device 104, or in the external device 110.

Additional physiological parameters may be determined using sensors of the catheter 102. For example, pressure may be measured (e.g., pressure in the right atrium), blood oxygenation may be measured, or presence (or absence) of a desired biological component may be determined. With a PICC or central venous catheter placed in a patient, such parameters may be determined by a nurse or other practitioner at a moment's notice without having to resort to more invasive and extensive procedures that might require admittance to a catheterization lab or to an intensive care unit.

Power and data may be transmitted to and from the sensors of the catheter 102 by a variety of techniques, including transmission along electrical conductors (e.g., wires or traces), wireless transmission such as described in any of the previously incorporated references, or wireless transmission such as described in U.S. Provisional Patent Application No. 63/342,812, entitled “Medical Devices, Systems and Related Methods” filed on May 17, 2022, the disclosure of which is incorporated by reference herein in its entirety, or by other appropriate techniques.

The use of “adapted to” or “configured to” herein is meant as open and inclusive language that does not foreclose devices adapted to or configured to perform additional tasks or acts. Additionally, the use of “based on” is meant to be open and inclusive, in that a process, act, calculation, or other action “based on” one or more recited conditions or values may, in practice, be based on additional conditions or values beyond those recited. Headings, lists, and numbering included herein are for ease of explanation only and are not meant to be limiting.

While the disclosed embodiments may be susceptible to various modifications and alternative forms, specific embodiments have been shown by way of example in the drawings and have been described in detail herein. It is noted that features, elements, or components of one embodiment may be combined with features, elements, or components of other embodiments without limitation. Thus, it should be understood that the invention is not intended to be limited to the particular forms disclosed. Rather, the invention includes all modifications, equivalents, and alternatives falling within the spirit and scope of the disclosure as defined by the following appended claims.

What is claimed is:

1. A medical device comprising:
a flexible, elongated member configured for insertion within a body, the flexible, elongated member having a proximal end, a distal end, and at least one lumen extending along a length of the flexible, elongated member;
a first temperature sensor disposed adjacent an exit port, the exit port being in fluid communication with the at least one lumen; and
a second temperature sensor disposed a known distance from the first temperature sensor.

2. The medical device of claim 1, further comprising at least one additional sensor.

3. The medical device of claim 2, wherein the at least one additional sensor comprises a pressure sensor.

4. The medical device of claim 1, wherein the flexible, elongated member is configured as at least one of a peripherally inserted central catheter (PICC) or a central venous catheter.

5. The medical device of claim 1, wherein the first temperature sensor and the second temperature sensor are in wireless electrical communication with an external device.

6. The medical device of claim 1, wherein the electrical communication of the first temperature sensor and the second temperature sensor includes providing power to the first temperature sensor and the second temperature sensor.

7. A method of determining cardiac output of a patient, the method comprising:
inserting a flexible, elongated member into a vein of a patient;
locating a distal end of the flexible, elongate member near a cavoatrial junction of the patient;
passing a fluid through a lumen of the flexible, elongated member and through an exit port of the flexible, elongated member;
determining a temperature of the fluid using a first sensor associated with the flexible, elongated member;
measuring a time period that it takes for a second sensor associated with the flexible, elongated member to record a temperature that correlates with the determined temperature of the fluid;
determining a flow characteristic based on a known distance between the first sensor and the second sensor and the measured time period.

8. The method according to claim 7, further comprising determining a fluid pressure using a third sensor associated with the flexible, elongated member.

9. The method according to claim 7, wherein inserting a flexible, elongated member into a vein of a patient includes inserting at least one of a peripherally inserted central catheter (PICC) and a central venous catheter into the vein of the patient.

10. The method according to claim 7, further comprising leaving the flexible, elongated member in the vein of the patient and repeating the acts of passing a fluid, determining a temperature, measuring a time period, and determining a flow characteristic.

11. The method according to claim 7, wherein leaving the flexible, elongated member in the vein of the patient includes leaving the flexible, elongated member in the vein of the patient for a period of at least a week.

12. A system comprising:
a medical device comprising:
a flexible, elongated member configured for insertion within a body such that a distal end may be positioned at or near a cavoatrial junction of a patient, the flexible, elongated member having a proximal end, and at least one lumen extending along a length of the flexible, elongated member;
at least one sensor coupled disposed at the distal end of the flexible, elongated member;
an external device in wireless communication with the medical device and configured to receive data from the at least one sensor.

13. The system of claim 12, wherein the at least one sensor includes at least one of a pressure sensor, a temperature sensor, a flow sensor, an oxygen sensor, and a glucose sensor.

14. The system of claim 12, wherein the at least one sensor includes a first temperature sensor and a second temperature sensor disposed a known distance from the first temperature sensor.

15. The system of claim 12, wherein the flexible, elongated member is configured as a peripherally inserted central catheter (PICC).

16. The system of claim 12, wherein the flexible, elongated member is configured as a central venous catheter (CVC).

17. The system of claim 12, wherein the external device provides power to the at least one sensor.

18. The system of claim 17, wherein the external device provides wireless power to the at least one sensor.

19. The system of claim 12, further comprising a volume of fluid in communication with lumen of the elongated, flexible member.

20. The system of claim 12, wherein the at least one sensor includes at least one temperature sensor and at least one pressure sensor.
